(19) United States
(12) Patent Application Publication
(10) Pub. No.: US 2013/0236517 A1
(43) Pub. Date: Sep. 12, 2013

(54) Apatite Forming Biomaterial

(71) Applicants: Leif Hermansson, Molle (SE); Fredrik Alpsten, Uppsala (SE)

(72) Inventors: Leif Hermansson, Molle (SE); Fredrik Alpsten, Uppsala (SE)

(73) Assignee: DOXA AB, Uppsala (SE)

(21) Appl. No.: 13/885,807

(22) PCT Filed: Nov. 16, 2011
(86) PCT No.: PCT/SE2011/051378

§ 371 Date: May 16, 2013

Related U.S. Application Data
(60) Provisional application No. 61/414,276, filed on Nov. 16, 2010.

(51) Int. Cl. A61L 24/02; A61L 24/00; A61K 6/033

(52) U.S. Cl. CPC A61L 24/02; A61K 6/033; A61L 24/001
USPC 424/400; 424/602

(57) ABSTRACT

The present invention relates to chemically bonded ceramic biomaterials, especially a dental material or an implant material. The main binder system forms a chemically bonded ceramic upon hydration thereof, and comprises powdered calcium aluminate and/or calcium silicate, and phase(s) to secure apatite formation at a pH close to neutrality. A second binder system—a cross-linking organic binder system which provides for initial crosslinking of the freshly mixed paste is advantageously added. The invention relates to a powdered composition for preparing the inventive chemically bonded ceramic biomaterial, and a paste from which the biomaterial is formed, as well as a kit comprising the powdered composition and hydration liquid, as well as methods and use of the biomaterial in dental and implant applications with the aim of remineralisation, integration and bone repair.

### BACKGROUND

In the art chemically bonded ceramics (CBCs)—as general materials and biomaterials—are known and have been described in a number of patent applications. Such materials are especially used in dental and orthopaedic applications. A number of requirements should preferably be fulfilled by such materials. The materials should be biocompatible. Other properties required of the paste forming the biomaterial, especially for dental applications, include good handling ability of the material with simple applicability in a cavity, moulding that permits good shaping ability, a hardening/solidification of the material that is sufficiently rapid for filling work without detrimental heat generation. After hardening, the resulting biomaterial formed should also provide serviceability directly following therapy, a high hardness and strength, corrosion resistance, adequate bonding between the hardened biomaterial and surrounding biological tissue, radio-opacity, good long time properties and good aesthetics of the resulting hardened material. The biomaterials may also comprise one or more additives, such as expansion compensating additives adapted to give the ceramic material dimensionally stable long-term attributes. For dental filling and cement materials it is preferred that the system comprises additives and/or is based on raw materials that contribute to translucency of the hydrated material.

WO 2005/039508 discloses a CBC system for dental and orthopaedic applications, which system has been developed to provide improved early-age properties and improved end-product properties, including bioactivity. The system includes two binding systems, a first initial working part-system, and second ceramic main system. The systems interact chemically. The main system is a cement-based system that comprises one or more CBCs selected from the group consisting of aluminates, silicates, phosphates, carbonates, sulphates and combinations thereof, having calcium as the major cation. The first binding system is based on a polycarboxylic acid, a co-polymer thereof, or a polycarboxylate (i.e. a salt or ester of a polycarboxylic acid), such as a polyacrylic acid and/or a salt thereof. The first binding system also requires the presence of metal ions such as Ca²⁺ for proper cross-linking thereof, and thus for obtaining the desired early-age properties of the overall system.

Biomaterials to be used in medical devices should be biocompatible and bioactive to facilitate remineralisation and integration with tissue. The present invention has been especially developed for formation of highly bioactive materials using formation of apatite phase(s) at a pH interval normally not considered possible. The finding according to the present invention is that apatite formation can be achieved at a pH interval of pH 5-8.

These findings refer in the first place to dental applications, and implants with high biocompatibility and bioactivity.

### SUMMARY OF THE INVENTION

The present invention relates to biomaterials system based on chemically bonded ceramics and a cross-linking acid system for preferably dental applications with a unique ability of forming apatite phases. The apatite formation according to the invention occurs in pH-ranges normally not considered to yield apatite. The biomaterial system includes a water-based liquid, a cross-linking polyacrylic system and a powder system, which powder system comprises an inorganic cement phase and slowly resorbable F-containing glass and/or a water soluble F-containing salt, contributing to the formation of a complex biomaterial containing one or more apatite phases. The invention relates to a powdered composition for preparing the inventive chemically bonded ceramic material, and a paste from which the material is formed, as well as a kit comprising the powdered composition and hydration liquid, as well as methods and use of the material in specified applications within odontology and orthopaedics.

The present invention relates to materials based on chemically bonded ceramics based on Ca-aluminate and/or Ca-silicate with apatite forming ability at pH intervals not considered to yield bioactivity. The material can be applied as powders and/or pastes.

The present invention relates to the formation of mixtures of and/or solid solutions of apatites. In the prior art, it has been a commonly accepted requirement that, in order for apatite to form, pH must be >8. The present inventors have surprisingly found that this requirement can be disregarded, and that full advantage of the solid solution features of apatite phases can be taken also at a lower pH interval (i.e. at a pH range of 5-8). The apatite phase is precipitated from the hydrating liquid and ions dissolved therein.

By using a powdered composition for preparing the chemically bonded ceramic bio-material, comprising a powdered inorganic cement, which cement comprises phases of Ca-aluminates and/or Ca-silicates, and slowly resorbable phases that contribute to ion release for formation of apatite phases, the biomaterials according to the present invention will exhibit apatite formation during the curing, and the end product will be bioactive.

### BRIEF DESCRIPTION OF THE DRAWINGS

FIG. 1, using scanning electron microscopy, shows a portion of a surface of the hydrated chemically bonded ceramic material substantially formed of apatite.

FIG. 2 shows an X-ray diffractogram of the surface, indicating the presence of apatite. The positions of two main hydroxyapatite (HA) peaks in the detected interval are marked by arrows.

### DETAILED DESCRIPTION OF THE INVENTION

The present invention is based on the surprising finding that apatite phases can be obtained during formation of biomaterials based on Ca-aluminate and/or Ca-silicate hydration already at pH ranges of 5-8. According to the present invention, this is possible in spite of the fact that, due to the presence of polyacrylic acids (PAA), the pH during curing and use of the ceramic paste containing water for hydration is below 8. The apatite formation is according to the present invention achieved by presence of additional slowly resorbable phases contributing to apatite formation. Apatite formation is a sign of the material to be bioactive, cf. ISO 23317. The presence of PAA, which will result in a pH<8, will thus surprisingly not prevent apatite formation, due the inventive selection of additional phases, which will yield ions which will contribute to solid solutions of apatite.

The formation of apatite, i.e. precipitation of apatite, occurs when the solubility product of apatite is reached. For the original hydroxyapatite the ions are Ca-ions, phosphate-ions and OH⁻-ions. This explains why a high pH (i.e. a high concentration of OH⁻) facilitates the apatite formation. According to the present invention, however, the solubility product of apatite can also be achieved by the presence of other ions, such as phosphates, carbonates, and earth metal ions, from slowly resorbable sources which are added to the main components forming the chemically bonded system, i.e. Ca-aluminates and/or Ca-silicates, and which ions, to some extent, can re-place the ionic species in the basic apatite structure, as will be described in more detail below. The OH-concentration, i.e. the requirement of a high pH value in the prior art, can thus be compensated for by the presence of other ions, and thus opens up for apatite formation at broad ex-tended pH-ranges.

A general chemical formula for solid solution apatite can be written as:

[(Me²⁺)ₓ(di- or trivalent anion)_{y}monovalent anion)_{z}]

In e.g. calcium hydroxyl apatite, Me is Ca²⁺, the di- or trivalent anion is PO₄³⁻, and the monovalent anion is OH⁻.

In contact with saliva and/or body liquid, wherein either of the ionic species CO₃²⁻ and PO₄³⁻ will be present, at least in low concentrations, a certain amount of either of the two di- and trivalent anions will automatically be added to the biomaterial from the saliva and/or body liquid. The phosphate anion can also be added to the original biomaterial via resorbable P-containing glasses, which will produce phosphate ions.

A P-containing resorbable glass will, at least to some extent, form PO₄³⁻ ions in aqueous solution.

Accordingly, any resorbable phase that will form either of the two species CO₃²⁻ and PO₄³⁻ in aqueous solution can also be used according to the invention.

According to the present invention the main components Ca-aluminate and/or Ca-silicate, and water will contribute to the monovalent anion OH⁻, and slowly resorbable phases may be added to provide extra contribution to the Me²⁺ position (e.g. Sr²⁺, Mg²⁺, Ba²⁺), in addition to the Ca²⁺ provided by the main components Ca-aluminate and/or Ca-silicate. Slowly resorbably phases may also be added to provide substitute anions for the monovalent anion position (e.g. F⁻, Cl⁻), which monovalent anions thus may substitute the OH⁻ anion in the monovalent anion position above.

Accordingly, the slowly resorbable phases according to the invention are added to provide ions which may occupy the different positions in the above general apatite formula, and which are therefore able to compensate for a high concentration of OH⁻.

Thus, slowly resorbable phases may be added to provide Cl⁻ and/or F⁻ ions for the monovalent anion position.

Slowly resorbable phases providing Sr²⁺, Mg²⁺, and/or Ba²⁺ ions for the Me²⁺ position, and slowly resorbable phases providing phosphate and/or carbonate ions for the di- or trivalent anion position may be used according to the invention.

For a number of the substitute ions, the resorbable phases can be in the form of a resorbable glass, such as for Sr²⁺, F⁻ and phosphate.

The presence of fluoride anions is believed to be crucial to the proper functioning of the invention. Fluoride anions can be provided by using a water soluble fluoride salt, e.g. SrF₂, LiF, KF, and NaF, or by using an F containing resorbable glass.

In order to for apatite to form already within the pH interval of 5-8, the F content of the powder should be at least 0.1% by weight of the powder, exclusive of any organic cross-linking polyacid present in the powder.

SrF₂ can also be added to provide Sr²⁺, which ions, when substituted in the Me²⁺ position will serve to provide radiopacity to the material formed.

Since the solubility product of apatite is very low (pKs approximately 10⁻⁵⁸), the added phases can be present in a low concentration, preferably, 1-20% by weight, and more preferably 4-17%. For a crystal to precipitate from a solution the ion concentration must be such that the solubility product can be reached. The solubility product, i.e. the point when apatite is precipitated, can thus be obtained even with a low OH-concentration. Thus, apatite can be formed even at a relatively low pH, in the range 5-8. Thus, as in the present application, an additional acid-based binding system can favourably be used, such as disclosed in e.g. WO 2005/039508.

In another aspect the invention relates to a paste obtained by mixing the powder composition with an aqueous hydration liquid based on water.

In a further aspect the invention relates to a kit comprising the powder and an aqueous hydration liquid based on water.

In another aspect the invention relates to a capsule mixing system containing the powder and an aqueous hydration liquid based on water.

According to the present invention highly biocompatible and even bioactive bio-materials can, in contradiction to the prior art general conception, be formed at pH-ranges close to neutrality. The invention is aimed at producing biomaterials for remineralisation, damaged bone substitute and bone ingrowth towards tissue, i.e. bone integration.

The present invention is preferably used as a dental luting cement, tooth fillings including underfillings, fissure sealings, and as endo products (including orthograde and retrograde fillings). The biomaterial according to the present invention is preferably used also for biomaterials for coatings of implants and for treatment of tissue and implants related to peri-implantitis due to the nanostructure of the biomaterial formed, as well as general bone void filling.

Bacteriostatic and antibacterial properties of the CBC material will be obtained when using a composition comprising a powdered inorganic cement phase based on calcium aluminate and/or calcium silicate phases selected from CA, C₁₂A₇, C₃A, C₂S, and C₃S, wherein the particles present in the powdered composition exhibit an average size of less than 10 μm, preferably a d(90)_{V} of less than 10 μm, and more preferably a d(99)_{V} of less than 10 μm. The powdered composition may additionally contain nano-porous inert filler particles of an average particle of less than 1 μm. Such nano-porous inert filler particles can be formed from hydration of CA, C₁₂A₇ and/or C₃A particles of an average size of less than 10 μm. The powdered composition should be essentially free from any calcium sulphate and calcium phosphate phases.

Accordingly, the apatite formation according to the present invention can be combined with antibacterial properties of the material at the same pH-range as proposed in the present invention. That is to say, according the present invention, the apatite forming material can be selected so at to also exhibit bacteriostatic and antibacterial properties.

Since the organic second binder system, which is based on an organic cross-linking polyacid, which is often provided in powder form, may be contained either in the powdered composition comprising the powdered inorganic cement phase, or may be contained in the hydration liquid, or in both, the organic cross-linking polyacid is excluded when calculating the percentages of weight of the constituents of the powdered system, especially F and resorbable phases.

### EXAMPLES

Description of raw materials and preparation:
a. The calcium aluminate (C₁₂A⁷=12(CaO)7(Al₂O₃)) used was synthesised and treated according to the description below.
b. Deionised water.
c. Poly acrylic acid, p.a. quality, having an average molecular weight in the interval 10,000-200,000.
d. Slowly resorbable glass A (Formulation A) of the composition SiO₂—CaO—BaO—P₂O₅—Na₂O—Al₂O₃ (in wt % 50-10-5-10-15-10) of an average particle size of 5 μm.
d. Slowly resorbable glass B (Formulation B) of the composition SiO₂—CaO—CaF₂—BaO—P₂O₅—Na₂O—Al₂O₃ (in wt % 50-5-10-5-10-10-10) average particle size 5 μm.
e. SrF₂.
f. Neutralised sodium Nitrilo Tri acetic Acid (Na₃—NTA) as pre-prepared standard.
g. Glass, inert, of the composition SiO₂—BaO—B₂O₃—Al₂O₃ (in wt % 50-30-10-10).

### Example 1

### Preparation of the Powder

The calcium aluminate used for this material was synthesised using high purity Al₂O₃ and CaCO₃. Appropriate amounts of the raw materials are weighed in to a suitable container (12:7 molar ratio). The powders are intimately mixed by tumbling in excess isopropanol. Thereafter, the isopropanol is removed, such as by evaporation of the solvent using an evaporator combining vacuum and heat and finally heating in oven. The next step is filling high purity Al₂O₃ crucibles with the powder mix and heat treating it above 1375° C. for 4 h. After heat treatment the material is crushed using a high energy crusher, in this case a roller crusher with alumina rollers. After crushing the calcium aluminate is milled using an air jet mill (Hosokawa Alpine) to the specified particle size distribution with a d(99)_{V} of <10 μm and an average particle size of 5 μm.

The final powder formulations A and B, respectively, are obtained in the following way: All powder components are weighed in with high accuracy according to the composition in Table 1 and in Table 2, respectively.

TABLE 1
Composition of the final powder Formulation A.

| | | Wt % (exclusive of |
|---|---|---|
| | | organic cross- |
| Raw material | Wt % | linking polyacid) |
| Calcium aluminate | 51.00 | 57.30 |
| C₁₂A₇ phase | | |
| Polyacrylic acid | 11.00 | N.A. |
| Tartaric acid | 2.00 | 2.25 |
| Glass (inert) | 27.50 | 30.90 |
| Ps < 0.5 μm | | |
| Resorbable glass A | 4.50 | 5.06 |
| SrF₂ | 4.00 | 4.49 |

TABLE 2
Composition of the final powder Formulation B.

| | | Wt % (exclusive of |
|---|---|---|
| | | organic cross- |
| Raw material | Wt % | linking polyacid) |
| Calcium aluminate | 50.00 | 56.18 |
| C₁₂A₇ phase | | |
| Polyacrylic acid | 11.00 | N.A. |
| Tartaric acid | 2.00 | 2.25 |
| Glass (inert) | 22.50 | 25.28 |
| Ps < 0.5 μm | | |
| Resorbable glass B | 10.50 | 11.80 |
| SrF₂ | 4.00 | 4.49 |

The components are weighed into a glass beaker, and the beaker is thereafter placed in a dry mixer and the components are mixed for 3 hours. The next step after mixing is sieving through a sieve in order to homogenise the powder and remove large agglomerates. After sieving, the powder is transferred to a suitable container, which is then sealed and stored dry. The powder is now ready for use.

### Example 2

### Preparation of the Liquid

0.35 wt % of Na₃—NTA was prepared. After the water has been added the bottle is shaken until all the salts have dissolved. The liquid is now ready for use.

### Example 3

### Description of Tests and Results Obtained

The powder and liquid described above including pure water were tested together in the below tests using a powder to liquid (P:L) ratio of 3.2:1.0 close to the w/c ratio for full con-version of the C₁₂A₇-phase. The material is mixed by hand using a spatula by bringing the required amount of powder and liquid on to a mixing pad and mixing them thoroughly for 40 seconds. Thereafter the paste was submerged in phosphate buffered saline (PBS) for a period of 2-30 days.

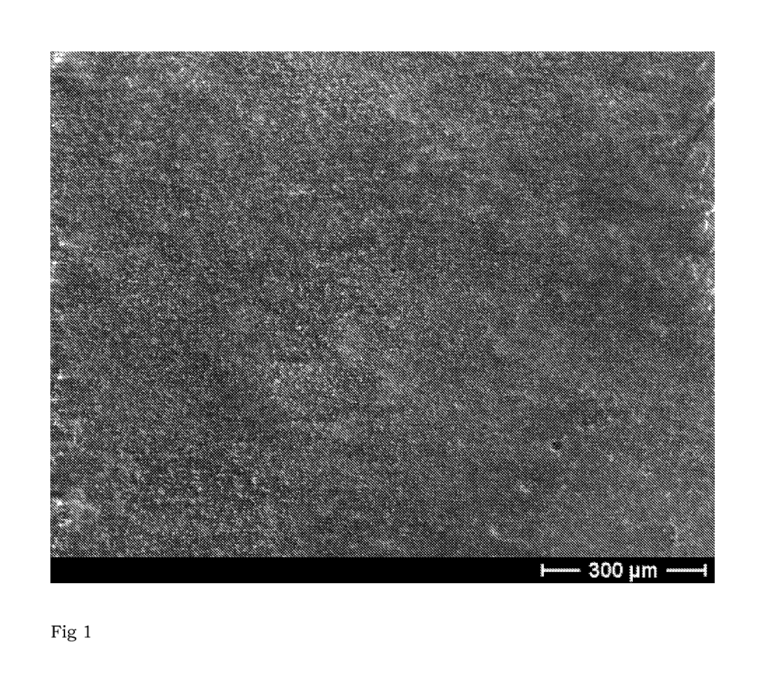

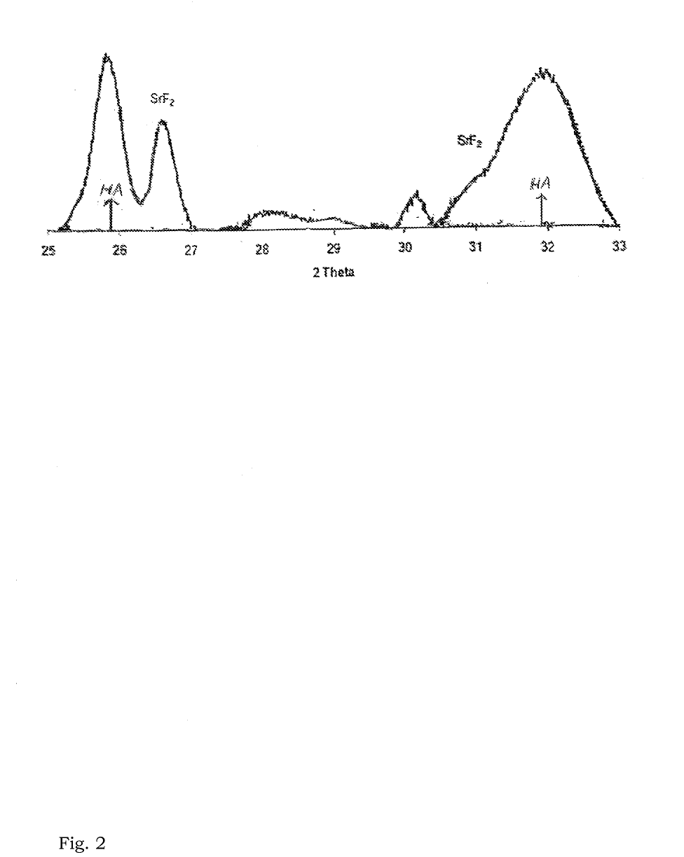

The bioactivity, defined as the ability of forming apatite has been shown by means of energy dispersive spectroscopy (EDS), scanning electron microscopy (SEM), transmission electron microscopy (TEM), grazing incidence X-ray diffraction (GI-XRD). The crystallised apatite is formed on the surface of the material. This occurs in spite of the pH value, which in all tests wherein PAA is present is below 8. The pH value of the pastes in Example 1 is 4.9 just after the preparation, and 7.1 after 48 hours. The exact apatite phase is difficult to establish, but with reference to FIG. 2, all data show for composition B (Table 2) the main apatite structure with small deviations from pure hydroxyl-apatite (HA—marked by arrows in the FIG. 2). FIG. 1 shows the formation of a new phase on the surface of the applied material, which material has a composition according to Table 2. FIG. 2. shows the phase formed on the surface in FIG. 1 to contain an apatite phase.

1. A powdered composition forming upon hydration thereof at ambient temperature a chemically bonded ceramic biomaterial containing apatite phases, which composition comprises a powdered inorganic cement phase based on calcium aluminate and/or calcium silicate phases wherein the powdered composition additionally contains a slowly resorbable F-containing phase selected from water soluble F-salts and F-containing resorbable glasses in an amount corresponding to at least 0.1% by weight of F, and, optionally, one or more additional resorbable phases selected from the following: resorbable phases containing Sr, Mg, Ba, and/or Cl, resorbable phases which are able to form CO₃²⁻ and PO₄³⁻ ions in aqueous solution, wherein the resorbable F-containing phase, and any additional optional resorbable phases are present in a total amount in the interval of 1-20% by weight of the powdered composition, as calculated exclusive of any organic, cross-linking polyacid present in the powdered composition.

2. The powdered composition of claim 1, wherein the slowly resorbable phase or phases comprise SrF₂, an F-containing resorbable glass, and/or a P-containing resorbable glass.

3. The powdered composition of claim 1, wherein the calcium aluminate and/or calcium silicate phases are selected from CA, C₁₂A₇, C₃A, C₂S, and C₃S, and in that the particles present in the powdered composition exhibit an average size of less than 10 μm, preferably a d(90)_{V} of less than 10 μm, and more preferably a d(99)_{V} of less than 10 μm.

4. The powdered composition of claim 3, additionally containing inert filler particles of an average particle of less than 1 μm, preferably the inert filler particles comprises nano-porous particles of hydrated Ca-aluminate.

5. The powdered composition of claim 3, which is free from calcium sulphate and calcium phosphate phases.

6. A paste formed from the powdered composition of claim 1, and an aqueous hydration liquid in a c/w ratio close to full hydration, i.e. full hydration±10% of the Ca-aluminate and/or Ca-silicate phases present, which paste additionally comprises a second, organic binder system based on an organic cross-linking polyacid.

7. The paste of claim 6, wherein the calcium aluminate and/or calcium silicate phases are present in an amount effective to provide an amount of at least 40% by volume of the calcium aluminate and/or calcium silicate phases in the resulting hydrated CBC biomaterial, more preferably at least 50% by volume.

8. The paste of claim 6, wherein the second binding system based on a cross-linking polyacid is included in an amount effective to provide an amount of 5-35% by volume of said second binding system in the resulting hydrated CBC biomaterial, preferably 10-25% by volume.

9. The paste of any one of claim 6, wherein nano-porous inert filler particles are present in an amount of less than 25% by volume.

10. A chemically bonded ceramic biomaterial containing apatite phases, formed from the powder of claim 1 upon hydration thereof by an aqueous hydration liquid.

11. A chemically bonded ceramic biomaterial containing apatite phases and exhibiting antibacterial properties, formed from the paste of claim 6 upon hydration thereof, having a nano-porous structure composed of hydrated crystals of a size within the range of 15-40 nm, and having a nanopore size and/or a pore channel width in the range of 1-4 nm.

12. The material of claim 11, wherein the number of nanopores including nanopore channels per square micrometer exceeds 500.

13. A kit comprising the powder of claim 1 and an appropriate amount of aqueous hydration liquid based on water, preferably providing an amount of water in a c/w ratio close to full hydration, i.e. to full hydration±10%.

14. The kit of claim 13 in the form of a capsule mixing system containing the powder and the aqueous hydration liquid based on water.

15. Use of the paste of claim 6 for remineralisation and/or bone repair, wherein the pH value can be essentially kept within an interval of 5-8.

16. Use of the paste of claim 6 for sealing an implant to another implant and/or to tooth or bone tissue wherein the pH value can be essentially kept within an interval of 5-8.

17. Use of the paste of claim 6 for cementation of a veneer to a tooth wherein the pH value can be essentially kept within an interval of 5-8.

18. Use of the paste of claim 6 as a tooth filling, in fissure sealing, in endo products, including orthograde and retrograde fillings wherein the pH value can be essentially kept within an interval of 5-8.

19. Use of the paste of claim 6 as a bone void filling biomaterial wherein the pH value can be essentially kept within an interval of 5-8.

20. Method of preparing a chemically bonded ceramic biomaterial forming apatite phases upon hydration thereof, comprising bringing a powder of claim 1 in intimate contact with an aqueous hydration liquid based on water, wherein the pH value is essentially kept within an interval of 5-8.
